(19) United States
(12) Patent Application Publication
(10) Pub. No.: US 2009/0090356 A1
(43) Pub. Date: Apr. 9, 2009

(54) Supralaryngeal Airway Including Instrument Ramp

(76) Inventor: Daniel J. Cook, St. Louis, MO (US)

Correspondence Address: LEWIS, RICE & FINGERSH, LC;ATTN: BOX IP DEPT., 500 NORTH BROADWAY, SUITE 2000, ST LOUIS, MO, 63102, US

(21) Appl. No.: 11/866,858

(22) Filed: Oct. 3, 2007

(51) Int. Cl. A61M 16/00

(52) U.S. Cl. 128/200.26; 128/207.15

(57) ABSTRACT

A supralaryngeal airway of the type used to facilitate lung ventilation and the insertion of endo-tracheal tubes or related medical instruments through a patient's glottis where the shield is constructed to include a protrusion which serves to direct instruments inserted through the airway away from the proximal base of the shield and into the glottis of the patient.

### BACKGROUND

1. Field of the Invention

The invention relates to an artificial airway device, more specifically to a supralaryngeal airway which includes a positioning device which assists in medical instruments, such as intubation tubes, passing from the airway into the laryngeal opening (glottis).

2. Description of the Related Art

In general, supralaryngeal airways such as laryngeal masks allowing for both rapid lung ventilation and the insertion of medical instruments and tubes into the glottis of patients have been described in patents, such as U.S. Pat. No. 4,509,514 to Brain and U.S. Pat. Nos. 6,422,239 and 5,937,860 to Cook the entire disclosures of which were herein incorporated by reference. Laryngeal masks generally consist of two major components, a breathing tube and a shield. These devices are inserted into a patient's throat, and when properly positioned, cover the glottis. A seal is generally formed around the circumference of the glottis by a ring-like structure located toward the front of the mask (patient end). Inflation or the shape of the ring exerts pressure against the front, sides, and rear portions of the oropharynx, securing the device in place such that the glottis is positioned in general alignment with a recessed cavity in the mask face. Extending from a point external to the oral cavity, the flexible breathing tube terminates within the recessed cavity, aligned axially with the glottis. The positioning of the flexible breathing tube allows the passage of endo-tracheal tubes or related medical instruments into the glottis, in addition to allowing for lung ventilation.

While current supralaryngeal airways such as laryngeal masks can provide for improved placement and breathing over a traditional endotracheal tube, they can still be improved. In particular, while the exit of the distal end of the respiratory tube in the recessed cavity of such a mask is generally aligned axially with the glottis, the curved pathway of the breathing tube and the natural shape and resilience of medical instruments such as tubes which may be inserted through the mask and into the glottis can result in the instrument extending into the cavity but missing the glottis in some cases.

When such a miss occurs, bruising of the structures surrounding the glottis area can occur which can result in damage. Also, it is possible for the instrument or tube to push past the ring and extend down into the esophagus, which can result in a medically dangerous situation where the intubation tube is not inflating the lungs, but instead is inflating the stomach.

### SUMMARY

The following is a summary of the invention in order to provide a basic understanding of some aspects of the invention. This summary is not intended to identify key or critical elements of the invention or to delineate the scope of the invention. The sole purpose of this section is to present some concepts of the invention in a simplified form as a prelude to the more detailed description that is presented later.

Because of these and other problems in the art, described herein is a supralaryngeal airway primarily intended to facilitate lung ventilation and the insertion of endo-tracheal tubes or related medical instruments into a patient's trachea as needed during general anesthesia, intensive care, critical patient care, or at any other time that ventilation would be desired. In a laryngeal mask embodiment of such a supralaryngeal airway, the mask comprises a flexible ventilation tube and a positioning shield generally conforming to the anatomy of the oropharynx region surrounding the glottis, and securely affixed to the distal end of the ventilation tube. The shield includes a protrusion which serves as a ramp to direct inserted instruments into the glottis when they are passed through the respiratory tube.

There is described herein, among other things, a supralaryngeal airway comprising; a respiratory tube having a distal end, a proximal end, and a length therebetween; and a shield comprising an outer ring and a posterior base, the posterior base forming the base of a recessed cavity and the outer ring surrounding said recessed cavity; wherein said distal end of said respiratory tube is connected to said shield via an entry point which allows fluid flow into said recessed cavity; and wherein said shield includes at least one protrusion within said recessed cavity, said protrusion being positioned so as to contact an instrument after said instrument passes through said respiratory tube and said entry point and direct said instrument away from said posterior base and into a glottis of a patient.

In an embodiment of the airway the protrusion includes two opposing sloped sides which slope may be between 0 and 90 degrees exclusive from the posterior base. In another embodiment of the airway the posterior base further includes a plurality of longitudinal ridges arranged within said recessed cavity.

There is also described herein, A supralaryngeal airway comprising; a respiratory tube having a distal end, a proximal end, and a length therebetween; a shield comprising an outer ring and a posterior base, the posterior base forming the base of a recessed cavity and the outer ring surrounding said recessed cavity; and a protrusion, said protrusion extending from said posterior base about 3 to about 10 millimeters, or, more preferably 5 to 8 millimeters, into said recessed cavity; wherein said distal end of said respiratory tube is connected to said shield via an entry point which allows fluid flow into said recessed cavity; and wherein said protrusion is positioned so as to contact an instrument after said instrument passes through said respiratory tube and said entry point and direct said instrument away from said posterior base and into a glottis of a patient.

In an embodiment of the airway the protrusion includes two opposing sloped sides which slope may be between 0 and 90 degrees exclusive from the posterior base. In another embodiment of the airway the posterior base further includes a plurality of longitudinal ridges arranged within said recessed cavity.

There is also described herein, the combination of a supralaryngeal airway and a human patient, the combination comprising; a human patient, the human patient having a glottis; a supralaryngeal airway, placed within said human patient, said supralaryngeal airway including: a respiratory tube having a distal end, a proximal end, and a length therebetween; a shield comprising an outer ring and a posterior base, the posterior base forming the base of a recessed cavity and the outer ring surrounding said recessed cavity; and a protrusion, said protrusion extending from said posterior base into said recessed cavity; wherein said distal end of said respiratory tube is connected to said shield via an entry point which allows fluid flow into said recessed cavity; and wherein said protrusion is positioned so as to contact an instrument after said instrument passes through said respiratory tube and said entry point and direct said instrument away from said posterior base and into said glottis of said patient.

In an embodiment of the airway the protrusion includes two opposing sloped sides which slope may be between 0 and 90 degrees exclusive from the posterior base. In another embodiment of the airway the posterior base further includes a plurality of longitudinal ridges arranged within said recessed cavity. The protrusion may be positioned to extend from the proximal base into the recessed cavity a distance of about 3 to about 10 millimeters, or about 5 to about 8 millimeters.

There is also described herein, a laryngeal mask comprising; a respiratory tube having a distal end, a proximal end, and a length therebetween; a shield comprising an outer ring and a posterior base, the posterior base forming the base of a recessed cavity and the outer ring surrounding said recessed cavity; an entry point connecting said respiratory tube to said shield in a manner which allows fluid communication from said respiratory tube to said recessed cavity; and means extending from said posterior base for contacting an instrument after said instrument passes through said respiratory tube and said entry point and directing said instrument away from said posterior base and into a glottis of a patient.

In an embodiment of the airway the means for contacting includes two opposing sloped sides which slope may be between 0 and 90 degrees exclusive from the posterior base. In another embodiment of the airway the posterior base further includes a plurality of longitudinal ridges arranged within said recessed cavity.

There is also described herein, a method of directing an instrument from a supralaryngeal airway into the glottis of a patient; the method comprising: providing a supralaryngeal airway, the airway including: a respiratory tube having a distal and a proximal end; a shield comprising an outer ring and a posterior base, said outer ring surrounding said posterior base in such as fashion as to create a recessed cavity; wherein said respiratory tube is connected to said shield in a fashion so as to be in fluid communication with said recessed cavity; forming a protrusion on said posterior base and internal to said recessed cavity; placing said airway in a throat of a patient so that said recessed cavity is adjacent a glottis of said patient; and feeding an instrument through said respiratory tube such that a portion of said instrument exits said distal end of said respiratory tube, contacts said protrusion, and is directed by said contact into said glottis of said patient.

In an embodiment of the method the protrusion includes two opposing sloped sides which slope may be between 0 and 90 degrees exclusive from the posterior base. In another embodiment of the airway the posterior base further includes a plurality of longitudinal ridges arranged within said recessed cavity. The instrument in any of the methods or devices may comprise an intubation tube.

### BRIEF DESCRIPTION OF THE DRAWINGS

FIG. 1 shows a perspective view of an embodiment of a partially assembled laryngeal mask including a shield and including a protrusion which serves as an instrument ramp.

FIG. 2 shows a top view of the embodiment of FIG. 1.

FIG. 3 shows a front view of the embodiment of FIG. 1

FIG. 4 shows a cut through along the line 4-4 of FIG. 2

FIG. 5 shows the same cut through as FIG. 4 with an intubation tube being passed through the respiratory tube, contacting the protrusion, and being directed away from the posterior base.

FIG. 6 shows an embodiment of how an embodiment of a laryngeal mask may be assembled.

FIG. 7 shows an embodiment of a shield prior to attachment of the respiratory tube.

FIG. 8 shows a respiratory tube and connector prior to attachment to a shield.

### DESCRIPTION OF THE PREFERRED EMBODIMENT(S)

The following detailed description illustrates by way of example and not by way of limitation. Described herein, among other things, is an embodiment of a supralaryngeal airway which includes a protrusion which serves as a ramp to direct tubes or other medical instruments which are passed through the airway to be better directed into the glottis. For purposes of this discussion, the medical instruments, tubes, or other structures which may be passed through the respiratory airway and into the glottis of a patient will be referred to as “instruments.” Such term being intended to be inclusive of any device so positioned and used.

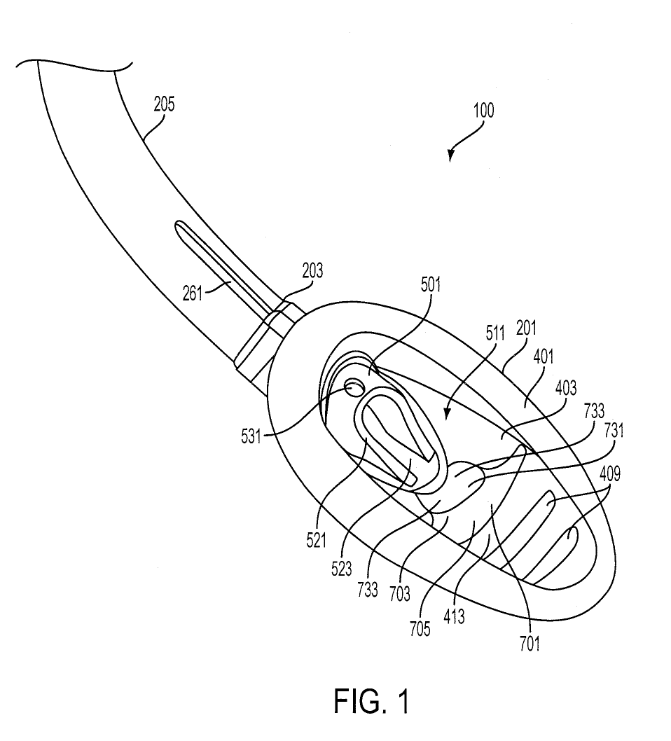

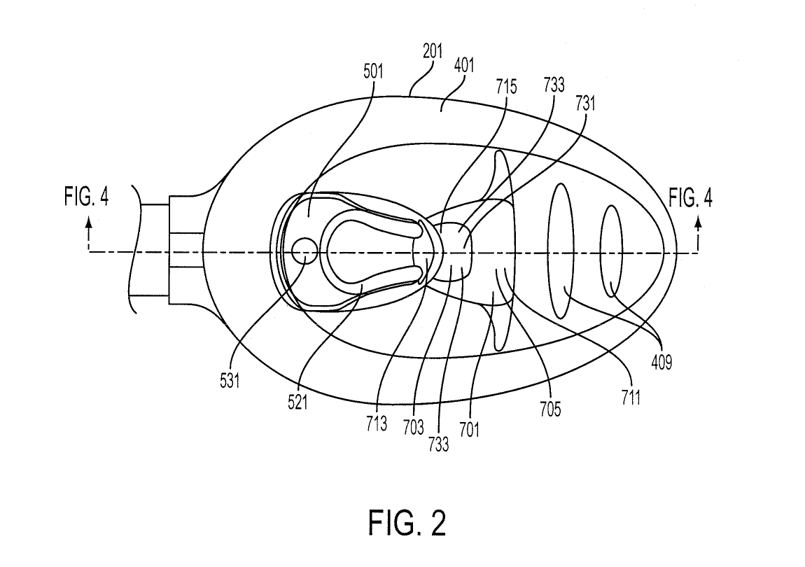

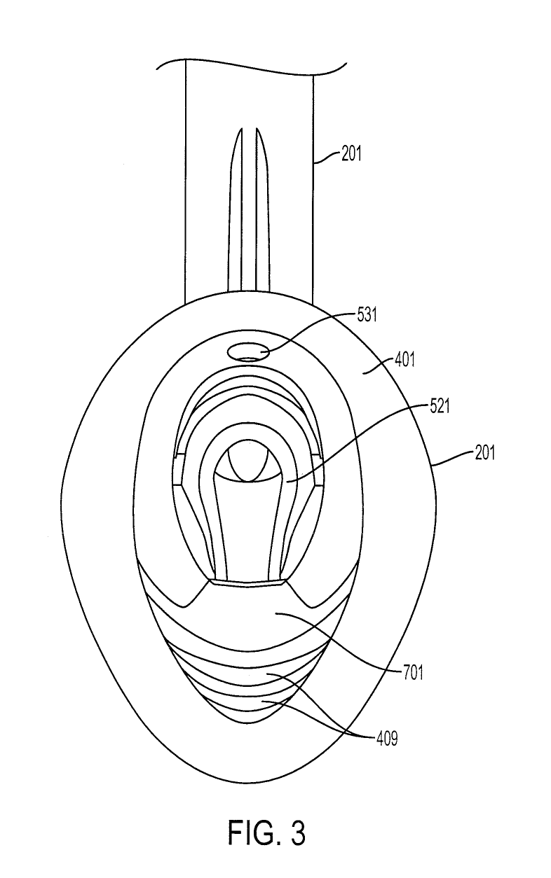

FIG. 1 provides for an embodiment of a supralaryngeal airway in the form of a removable laryngeal mask airway (100). This mask (100) is chosen as an exemplary form of supralaryngeal airway to simply demonstrate how the protrusion and methods of operating for insertion of instruments as discussed herein can be accomplished. It is in no way intended to be descriptive of all airways which may be used in other embodiments. In the depicted embodiment, the laryngeal mask (100) generally comprises two major components. There is a positioning shield (201) which in the depicted embodiment is inflatable. The shield (201) is secured toward the distal end (203) of a respiratory tube (205) which is formed into an arcuate curve. The laryngeal mask (100) is generally composed of a relatively soft flexible material such as, but not limited to, silicone-rubber polymer or plastics. The laryngeal mask (100) may also include inflation apparatus for inflating at least a portion of the shield to maintain the mask's (100) position in the patient. However, as the manner and construction of such inflation technologies do not affect the current invention, those components are not discussed in detail herein.

The positioning shield (201) comprises a generally wedge-shaped ellipsoid, ovoid, or toroid outer ring (401) with a pliable molded posterior base (403) attached thereto so that the posterior base (403) forms the base of, and the outer ring (401) surrounds, a recessed cavity (511). The outer ring (401), in an embodiment, may be repeatedly inflatable and deflatable. The outer ring (401) of such an embodiment, when inflated, is sized and shaped to generally conform to the approximate available space in the oropharynx region. In an alternative embodiment, the outer ring (401) need not be inflatable but may use its size and structure to fill the available space.

The posterior base (403) is secured longitudinally within the hole in the center of the outer ring (401). The posterior base (403) is generally attached in a fashion to form an elongated and tapered hemisphere relative the generally major plane of the outer ring (401) so as to give the shield (201) an overall shape such as that seen in the FIGS. In the depicted embodiment, there are semi-rigid raised ridges (409) positioned longitudinally parallel to each other along the surface (413) of the posterior base (403) “inside” the hole of the outer ring (401).

The shield (201) is generally connected to the respiratory tube (205) by means of a hollow connector (501) which allows the respiratory tube (205) to pass through the shield (201) and into the recessed cavity (511) formed above the posterior base (403) and inside the “hole” of the outer ring (401). In a preferred assembly, the hollow connector (501) is attached generally to the distal end (203) of the respiratory tube (205) and may comprise a somewhat more rigid construction than the other components. The connector (501) may be inserted into the end of the respiratory tube, or may be molded as one monolithic piece in an alternative embodiment. The connector (501) preferably includes a groove (551) which is circumferentially arranged toward the distal end (203) of the respiratory tube (205) and a raised disk (553) placed distal of the groove (551) but proximal the rest of the connector (501).

The respiratory tube (205) passes through the proximal end (505) of the inflatable positioning shield (201). The connector (501) therefore gives an access into the shield recess (511) from the interior of the respiratory tube (205) allowing air to pass from the recessed cavity (511) into the distal end (203) of the respiratory tube (205) and from there out the proximal end (207) of the respiratory tube (205). The distal end of the connector (501) is generally formed into an angle (521) to the length of the respiratory tube (205) which is generally between 0 and 90 degrees and preferably about 30 to about 35 degrees with the posterior base (403), forming an elongated elliptically shaped distal lumen (523) open to the interior of the shield recess (511) and interior of the respiratory tube (205).

There may also be included a ventilation lumen (531) through the connector (501) to provide an alternate airway in the event the distal lumen (523) becomes obstructed during patient lung ventilation. The ventilation lumen (531) also generally prevents the formation of a pressure differential between the recessed cavity (511) and flexible respiratory tube (205). Absent a pressure differential, any object obstructing the distal lumen (523) will not generally become inextricably lodged.

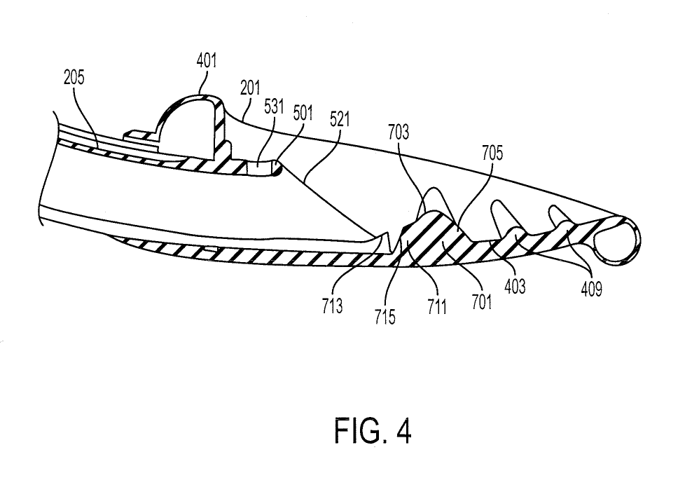

The shield (201) recess (511) will also generally include a protrusion (701). This protrusion (701) is generally positioned outside of the respiratory tube and toward the distal end of the connector (501) and serves to form a “ramp” on the posterior base (403) of the shield. The protrusion (701) will generally be formed, as in FIG. 4 to have two sloping sides (703) and (705). The first of the sloping sides (703) being arranged toward the connector (501) while the second (705) is toward the distal end of the shield (201). The sloping sides (703) and (705) are preferably smoothly sloping and there are no abrupt points of slope change. In an embodiment, the first sloping side (703) may be arranged to have a slightly trough-like or recessed structure where the center (731) of the sloping side (703) is lower than the outsides (733). This structure can serve to realign a slightly offset instrument upon the instrument passing through the entry point.

Depending on the positioning of the protrusion (701) the protrusion (701) may comprise a number of different sizes and elevations. In the depicted embodiment, the protrusion will preferably extend from about 3 to about 10 millimeters, more preferably from about 5 to about 8 millimeters, from the posterior base (403). However, the protrusion (701) need not be placed at the specific location shown in FIGS. and its elevation may change depending on the positioning. As the glottis of a patient will generally be located toward the distal end of the shield (201), moving the protrusion (701) toward the distal end will generally require it to be taller. Moving it towards the connector (501) or respiratory tube (205) will allow it to be shorter. In both instances, the change in elevation allows for the instrument to be directed into the glottis by contact with the protrusion (701). The protrusion will serve as one means for directing the instruments into the glottis of the patient.

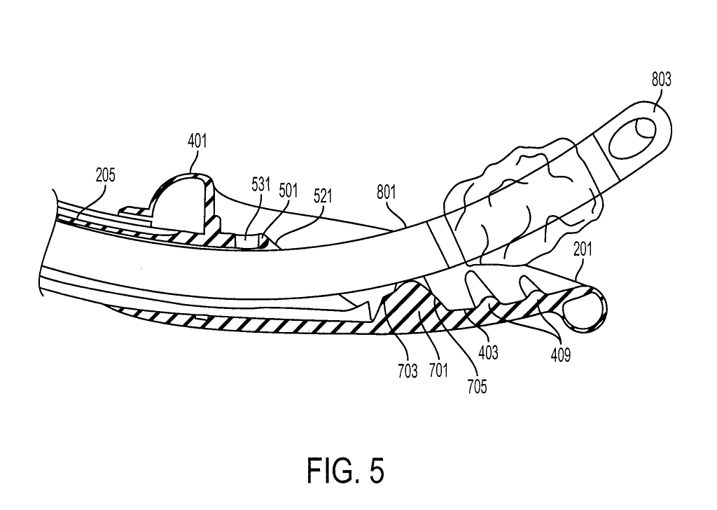

The first slope (703) which is toward the connector (501), serves to provide a ramp for instruments which are to be inserted through the glottis of a patient via the mask (100) to be directed up and away from the posterior of the shield (401) as they exit the connector (501). In particular, as shown in FIG. 5, an instrument (801) is placed inside the respiratory tube (205) and pushed down toward the shield (201). The instrument's end (803) then contacts the protrusion (701) upon exiting the distal end (203) of the respiratory tube (205) and will be directed up and away from the posterior base (403) of the shield (201) by the interaction. In particular, the end (803) of the instrument (801) will contact the sloped surface (703) and be directed upward by interaction with the surface (703). As the instrument (801) will have length and extend back into the respiratory tube (205), the protrusion (701) will operate in conjunction with the respiratory tube (205) and the upper surface of the connector (501) to provide for an angled departure of the instrument (801) from the posterior base (403). Specifically, the instrument will generally have a relatively limited angle of departure from the base being “pinched” between the protrusion (701) and connector (501) as shown in FIG. 5.

It is preferred that the second side (705) of the protrusion (701) also include a sloped surface. The second side's sloped surface (705) serves to provide for a directional surface for the epiglottis to slide along when the mask (100) is inserted. As discussed above, the shield (201) may include a plurality of semi-rigid raised ridges (409) which, in an embodiment, may, in part, serve to assist insertion by directing the epiglottis along the shield (201) and onto the top of the connector (501) (toward the ventilation lumen (531)). By having a second sloped surface (705) on the protrusion, the epiglottis generally will not become wedged behind the protrusion (701) during insertion of the mask (100) and instead the epiglottis will be directed upward over the protrusion (701) and onto the angle (521) of the connector (501). The epiglottis will then continue to contact the angle (521) of the connector (501) and eventually be positioned generally on top of the connector (501), generally in the position of the ventilation lumen (531).

The respiratory tube (205) may be formed in any manner known to those of ordinary skill in the art but will generally form a smoothly curving hollow cylinder of generally circular or elliptical cross-section preferably approximating, for ease of insertion, the shape of the human throat. The respiratory tube (205) is preferably sized and shaped to accommodate the passage of instruments such as, but not limited to, endo-tracheal tubes and related medical devices up to 9.0 French in diameter. The length of respiratory tube (205) is such that when the laryngeal mask (100) is properly positioned for use within the oropharynx, the attachment (proximal) end (207) of respiratory tube (205) is located exterior to the oral cavity of the patient. The attachment end (207) of the respiratory tube (205) terminates in an unobstructed proximal lumen (209), providing a direct pathway through the respiratory tube (205) to the distal end (203) and distal lumen (523). In alternative embodiments, the attachment end (207) may be fitted with a removable adapter (871) suitable for connection to a variety of medical devices, for example, lung ventilation machines.

There may be included on the respiratory tube (205), in some embodiments, a grooved recess (261) placed on the inside curve of the respiratory tube (205) or elsewhere. This grooved recess (261) allows for an inflation tube (not shown) to be placed in more defined contact with the respiratory tube (205) and also be connected to the shield (201) so as to inflate it. In alternative embodiments, alternative shield (201) structures may be used which may eliminate the inflation tube or place it in an alternative position.

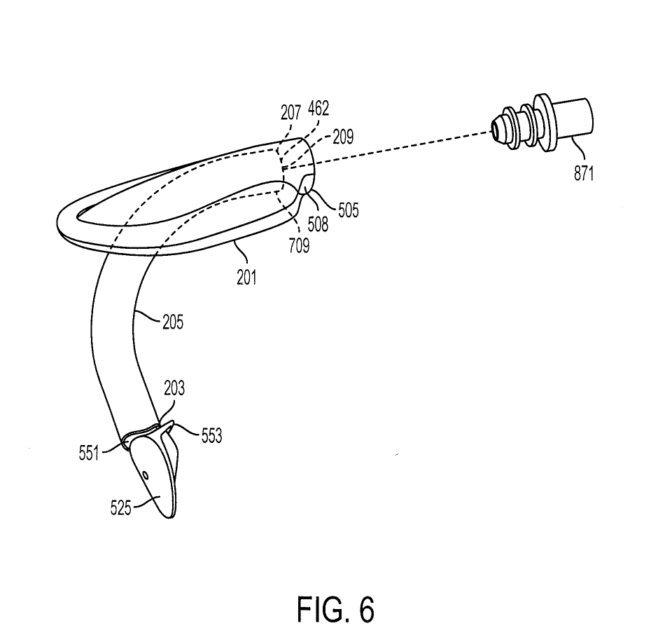

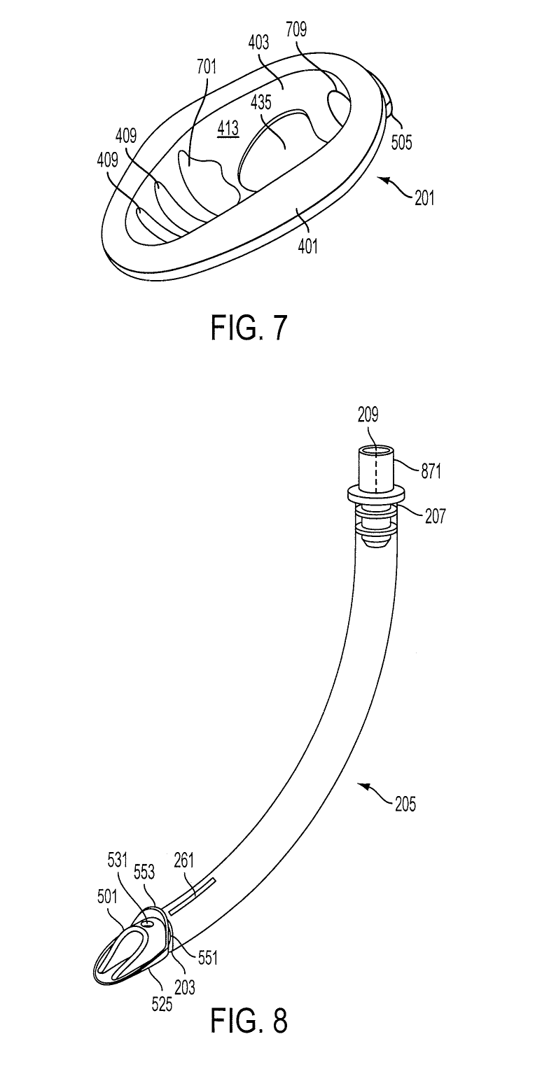

In an embodiment, in order to assemble the mask (100), it is generally preferred that the following steps be performed to assemble the components, generally as indicated in FIG. 6. The connector (501) will be attached to the distal end of the respiratory tube (205) as shown in FIG. 8. The shield (201) will have a hole (709) through the proximal wall of the recess (511) as shown in FIG. 6. As should be apparent from FIG. 6, the hole (709) therefore presents a relatively straight passage which extends from inside the recess (511) through the outer ring (401) and out the proximal end (505) of the shield (201). There is also generally formed a connection recess (435) in the posterior base (403). This may be cut out or may be formed by simple compression of the material forming the posterior base (403).

The respiratory tube (205) is now inserted, proximal end (207) first, through the hole (709) and passed through the outer ring (401) thus extending from the posterior end (505) of the shield (201). The hole (709) and entry point (462) are preferably sized and shaped to be of relatively similar size to the exterior diameter of the respiratory tube (205) so that a tight connection is formed by the respiratory tube (205) distending the material of the shield (201) slightly in both places. The respiratory tube (205) will continue to be slid through the hole (709) until the hole (709) interacts with the groove (551) and disk (553). At that point, the connector (501) and disk (553) will generally be the only portions of the respiratory tube (205) which has not passed through the hole (709) and the reinforced support (525) will be adjacent to the recess (435) in the posterior base (403). These pieces will then be connected together resulting in the connector (501) being positioned in the recess (511) and generally flush with the interior of the posterior base (403). The connection between the posterior base (403) and the reinforced support (525) may be formed in any manner known to one of ordinary skill in the art, however, in a preferred embodiment, the two devices are adhered together with a generally non-separable adhesive. The respiratory tube (205) now is arranged to generally pass through the outer ring (401).

In the depicted embodiment, the disk (553) and groove (551) formed toward the distal end (203) of the respiratory tube (205) serve to reinforce the connection between the connector (501) and the ring (401). In particular, the material surrounding the hole (709) will end up being stretched by the passing of the respiratory tube (205) until the hole (709) is aligned with the groove (551). The material will then relax and the hole (709) will collapse slightly into the groove (551). This provides a first level of sealing. The disk (553) adjacent to the hole (709) can then be provided with an adhesive which adheres to the shield (201) forming the seal (703).

The protrusion (701) will generally be formed, at least in part, during the formation of the shield (401). In the depicted embodiment, a first portion (711) of the protrusion is formed as part of the shield (401), as shown in FIG. 4, slightly separated from the end of the connector to provide for easier placement of the connector (501). A second portion (713) of the protrusion (701) is constructed on the distal end of the connector (501) which results in the first sloped side (703) including a channel (715) after the connector (701) is positioned. In an embodiment, this channel (715), to provide for manufacturing ease, is filled during assembly. In an embodiment, the channel (715) is filled with the same adhesive used to secure connector (501) in recess (515), the adhesive may be applied so as to cover not just the channel (715) but to also coat all or a portion of the first portion (711) of the protrusion (701) or the second portion (713) to provide for the smooth surface. In a still further embodiment, the first portion (711), second portion (713), or the entire protrusion (701) may be formed by applying adhesive to the connector and/or shield (401) to form the protrusion (701) in the desired shape and size.

While in the above embodiment as depicted in FIG. 6, the proximal end (207) of the respiratory tube is first threaded through the hole (703), in an alternative embodiment, the connector (501) and respiratory tube (403) may actually be inserted in the opposing direction to the embodiment shown in FIG. 6. In this alternative embodiment, the connector (501) may be attached once the respiratory tube (205) is placed on the connector (501) and the distal end (203) of the respiratory tube (205) may be first inserted through the outer ring (401) exiting the hole (703) in the recess (511). While this method is viable in most cases, it is generally not preferred as it is usually more difficult to perform.

In another embodiment, the connector (501) and/or respiratory tube (205) need not pass through the outer ring (401) to access the recessed cavity (511). Instead, the respiratory tube (205) and/or connector (501) may pass through the posterior base (403) or other component of the shield (401) in such a manner as to allow fluid flow between the respiratory tube (205) and the recessed cavity (511).

Generally, use of the laryngeal mask (100) would proceed as follows. The mouth of the patient is opened and their head positioned for insertion of the mask (100). The outer ring (401) is pushed into the oropharyngeal region. The smooth arcuate curves of the combined respiratory tube (205) and shield (201) positions the laryngeal mask (100) in alignment with the glottis. The epiglottis will contact the ridges (409) if present and the second sloped surface (705) of the protrusion (701) and be directed by contact with the surface (705) on to the angle (521) of the connector (501). It will continue up angle (521) until in proximity with the ventilation lumen (531). Upon proper positioning, as generally determined by a resistance to further forward motion, the airway is ready for use.

Positioned within the recessed cavity (511), the distal lumen (523) is axially aligned with the glottis, permitting lung ventilation to be performed, or allowing instruments inserted through the respiratory tube (205) to exit through the distal lumen (523). Upon passing through the distal lumen (523) such instrument will contact the first surface (703) of the protrusion (701) and the instrument will be directed up and away from the shield (401) and directly toward the glottis, facilitating the instrument being directed into the glottis. Removal of the laryngeal mask (100) is normally the reverse of the insertion procedure described above.

While the invention has been disclosed in connection with certain preferred embodiments, this should not be taken as a limitation to all of the provided details. Modifications and variations of the described embodiments may be made without departing from the spirit and scope of the invention, and other embodiments should be understood to be encompassed in the present disclosure as would be understood by those of ordinary skill in the art.

1. A supralaryngeal airway comprising;
a respiratory tube having a distal end, a proximal end, and a length therebetween; and
a shield comprising an outer ring and a posterior base, the posterior base forming the base of a recessed cavity and the outer ring surrounding said recessed cavity;
wherein said distal end of said respiratory tube is connected to said shield via an entry point which allows fluid flow into said recessed cavity; and
wherein said shield includes at least one protrusion within said recessed cavity, said protrusion being positioned so as to contact an instrument after said instrument passes through said respiratory tube and said entry point and direct said instrument away from said posterior base and into a glottis of a patient.

2. The airway of claim 1 wherein said protrusion includes two opposing sloped sides.

3. The airway of claim 2 wherein said slope is between 0 and 90 degrees exclusive from the posterior base.

4. The airway of claim 1 wherein said posterior base further includes a plurality of longitudinal ridges arranged within said recessed cavity.

5. A supralaryngeal airway comprising;
a respiratory tube having a distal end, a proximal end, and a length therebetween;
a shield comprising an outer ring and a posterior base, the posterior base forming the base of a recessed cavity and the outer ring surrounding said recessed cavity; and
a protrusion, said protrusion extending from said posterior base about 3 to about 10 millimeters into said recessed cavity;
wherein said distal end of said respiratory tube is connected to said shield via an entry point which allows fluid flow into said recessed cavity; and
wherein said protrusion is positioned so as to contact an instrument after said instrument passes through said respiratory tube and said entry point and direct said instrument away from said posterior base and into a glottis of a patient.

6. The airway of claim 5 wherein said protrusion includes two opposing sloped sides.

7. The airway of claim 6 wherein said slope is between 0 and 90 degrees exclusive from the posterior base.

8. The airway of claim 5 wherein said posterior base further includes a plurality of longitudinal ridges arranged within said recessed cavity.

9. The airway of claim 5 wherein said protrusion extends from said posterior base about 5 to about 8 millimeters into said recessed cavity.

10. The combination of a supralaryngeal airway and a human patient, the combination comprising;
a human patient, the human patient having a glottis;
a supralaryngeal airway, placed within said human patient, said supralaryngeal airway including:
a respiratory tube having a distal end, a proximal end, and a length therebetween;
a shield comprising an outer ring and a posterior base, the posterior base forming the base of a recessed cavity and the outer ring surrounding said recessed cavity; and
a protrusion, said protrusion extending from said posterior base into said recessed cavity;
wherein said distal end of said respiratory tube is connected to said shield via an entry point which allows fluid flow into said recessed cavity; and
wherein said protrusion is positioned so as to contact an instrument after said instrument passes through said respiratory tube and said entry point and direct said instrument away from said posterior base and into said glottis of said patient.

11. The airway of claim 10 wherein said protrusion includes two opposing sloped sides.

12. The airway of claim 11 wherein said slope is between 0 and 90 degrees exclusive from the posterior base.

13. The airway of claim 10 wherein said posterior base further includes a plurality of longitudinal ridges arranged within said recessed cavity.

14. The airway of claim 10 wherein said protrusion extends into said recessed cavity a distance of about 3 to about 10 millimeters.

15. The airway of claim 14 wherein said protrusion extends into said recessed cavity a distance of about 5 to about 8 millimeters into said recessed cavity.

16. A laryngeal mask comprising;
a respiratory tube having a distal end, a proximal end, and a length therebetween;
a shield comprising an outer ring and a posterior base, the posterior base forming the base of a recessed cavity and the outer ring surrounding said recessed cavity;
an entry point connecting said respiratory tube to said shield in a manner which allows fluid communication from said respiratory tube to said recessed cavity; and
means extending from said posterior base for contacting an instrument after said instrument passes through said respiratory tube and said entry point and directing said instrument away from said posterior base and into a glottis of a patient.

17. The mask of claim 16 wherein said means for contacting includes two opposing sloped sides.

18. The mask of claim 17 wherein said slope is between 0 and 90 degrees exclusive from said posterior base.

19. The airway of claim 16 wherein said posterior base further includes a plurality of longitudinal ridges arranged within said recessed cavity.

20. A method of directing an instrument from a supralaryngeal airway into the glottis of a patient; the method comprising:
providing a supralaryngeal airway, the airway including
a respiratory tube having a distal and a proximal end;
a shield comprising an outer ring and a posterior base, said outer ring surrounding said posterior base in such as fashion as to create a recessed cavity;
wherein said respiratory tube is connected to said shield in a fashion so as to be in fluid communication with said recessed cavity;
forming a protrusion on said posterior base and internal to said recessed cavity;
placing said airway in a throat of a patient so that said recessed cavity is adjacent a glottis of said patient; and
feeding an instrument through said respiratory tube such that a portion of said instrument exits said distal end of said respiratory tube, contacts said protrusion, and is directed by said contact into said glottis of said patient.

21. The method of claim 20 wherein said protrusion is formed to include two opposing sloped sides.

22. The method of claim 21 wherein said slope is between 0 and 90 degrees exclusive from said posterior base.

23. The method of claim 21 further including forming on said posterior base a plurality of longitudinal ridges arranged within said recessed cavity.

24. The method of claim 20 wherein said instrument comprises an intubation tube.
